(19) United States
(12) Patent Application Publication
(10) Pub. No.: US 2011/0311452 A1
(43) Pub. Date: Dec. 22, 2011

(54) INFLAMMATION TARGETING PARTICLES

(75) Inventors: Mauro Ferrari, Houston, TX (US); Rita Serda, Pearland, TX (US)

(73) Assignee: BOARD OF REGENTS OF THE UNIVERSITY OF TEXAS SYSTEM, Austin, TX (US)

(21) Appl. No.: 13/141,475

(22) PCT Filed: Dec. 23, 2008
(86) PCT No.: PCT/US2008/014001

§ 371 Date: Sep. 8, 2011

(51) Int. Cl. A61K 49/00; A61P 35/00; A61P 29/00; A61K 9/14; A61P 9/00; B82Y 15/00; B82Y 5/00

(52) U.S. Cl. 424/9.1; 424/489; 977/700; 977/906; 977/927

(57) ABSTRACT

Opsonizable micro- or nanoparticles, that contain at least one active agent, such as an imaging or therapeutic agent; that have a positive surface charge and that do not contain on their surface targeting ligands, such as antibodies, peptides or aptamers, can be used to treating and/or monitoring a condition associated with an inflammation, such as a cytokine stimulated inflammation.

### STATEMENT FOR FEDERALLY FUNDED RESEARCH

Some research underlying the invention has been supported by federal funds from under grants nos. W81XWH-07-1-0596 and DoD TATRC W81XWH-07-2-0101. The U.S. government may have certain rights in this invention.

### FIELD

The present disclosure generally relates to vehicles for delivery active agents, such as a therapeutic agent or an imaging agent and, in particular, to micro or nanoparticles capable to target inflammation.

### SUMMARY

According to one embodiment, a method for treating or monitoring a condition associated with an inflammation, comprises administering to a subject in need thereof a composition comprising opsonizable micro- or nanoparticles, that contain at least one active agent, wherein a surface of the micro or nanoparticles a) has a positive electrical charge and b) does not contain targeting ligands.

According to another embodiment, a composition comprises opsonizable micro- or nanoparticles, that contain at least one active agent, wherein a surface of the micro or nanoparticles a) has a positive electrical charge and b) does not contain targeting ligands. Yet according to another embodiment, a method for targeting inflamed cells in a subject, comprises administering to the subject a composition comprising opsonizable micro- or nanoparticles, that contain at least one active agent, wherein a surface of the micro or nanoparticles a) has a positive electrical charge and b) does not contain targeting ligands.

### DRAWINGS

FIGS. 1A-D relate to uptake of oxidized, APTES, or PEGylated silicon particles by Human Umbilical Vein Endothelial Cells (HUVECs) and J774 macrophage cells. FIG. 1A presents Scanning electron micrographs of serum-free internalization of 3.2 μm silicon particles by HUVECs. Left images have a resolution bar of 5 pm; right images have a resolution bar of 2 μm. FIG. 1B is a diagram that compares internalization by HUVECs between serum free and opsonized particles after 1 hour incubation at 37° C. FIGS. 1C is a Table presenting electrostatic (zeta) potential of 3.2 μm microparticles before and after serum opsonization (100% serum for 60 min, 4° C.). FIG. 1D demonstrates an impact of serum on uptake of 1.6 μm particles by J774 macrophage (*p<0.03) after 1 hour incubation at 37° C. Y-axis in FIGS. 1B and 1D is the percentage of cells with particles (high side scatter cells).

FIGS. 2A-C relate to uptake of IgG opsonized silicon particles by HUVEC cells and J774 macrophage cells. FIGS. 2A and 2B present results of flow cytometry analysis of uptake by HUVEC (A) and J774 (B) cells serum-free vs IgG-opsonized 3.2 μm oxidized microparticles after 1 hr incubation at 37° C. FIG. 2C presents quantitative surface expression of FCγRs determined by flow cytometric analysis.

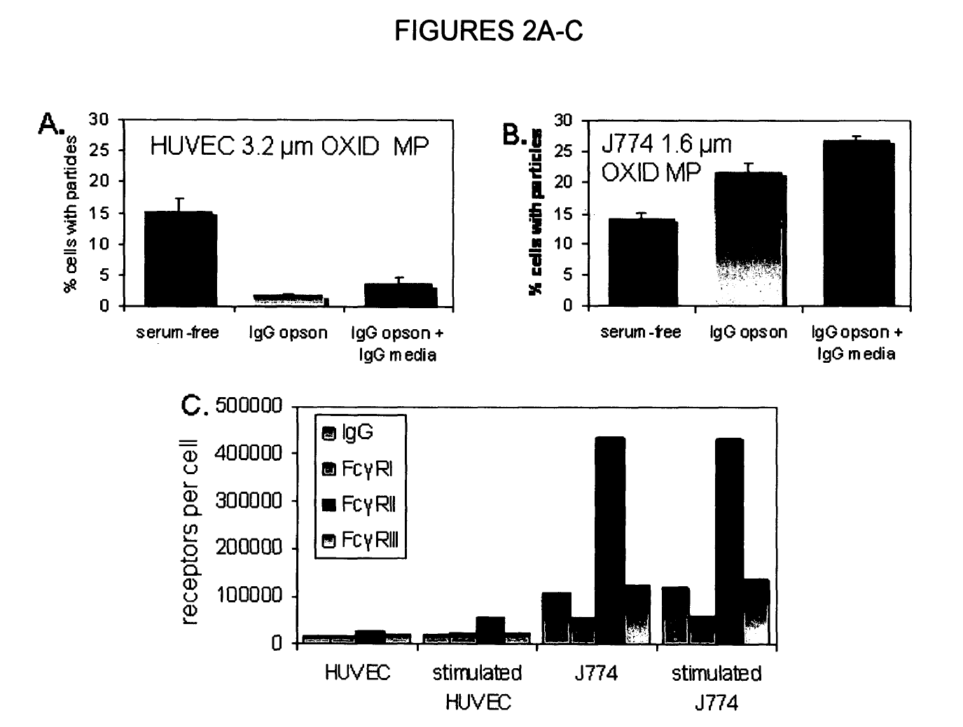

FIGS. 3A-D relate to uptake of silicon particles by cytokine stimulated HUVEC cells and J774 macrophage cells. FIG. 3A is a diagram that compares an uptake of oxidized, APTES, and PEGylated 3.2 μm silicon particles between control HUVEC cells and cytokine-stimulated HUVEC cells. FIG. 3B is a diagram that compares an uptake of oxidized, APTES, and PEGylated 3.2 μm silicon particles between control J774 cells and cytokine-stimulated J774 cells. FIG. 3C and 3D are scanning electron micrographs of 3.2 μm silicon particle uptake by HUVEC (C) and J774 (D) cells (30 min incubation at 37° C.).

FIGS. 4A-C relate to internalization of oxidized silicon particles by HUVECs (serum-free). FIG. 4A shows scanning electron micrographs of HUVECs grown on silicon chips after incubation with either 1.6 μm, 3.2 μm, or both sizes of oxidized silicon particles at 37° C. for 15 min, 30, or 60 min. FIG. 4B shows confocal micrographs of HUVECs incubated with 3.2 μm oxidized silicon microparticles for 15 and 120 min at 37° C. using Alexa Fluor 555 Phalloidin for actin staining. FIG. 4C shows confocal projection images cropped through the center to illustrate particle location at either 60 or 120 min.

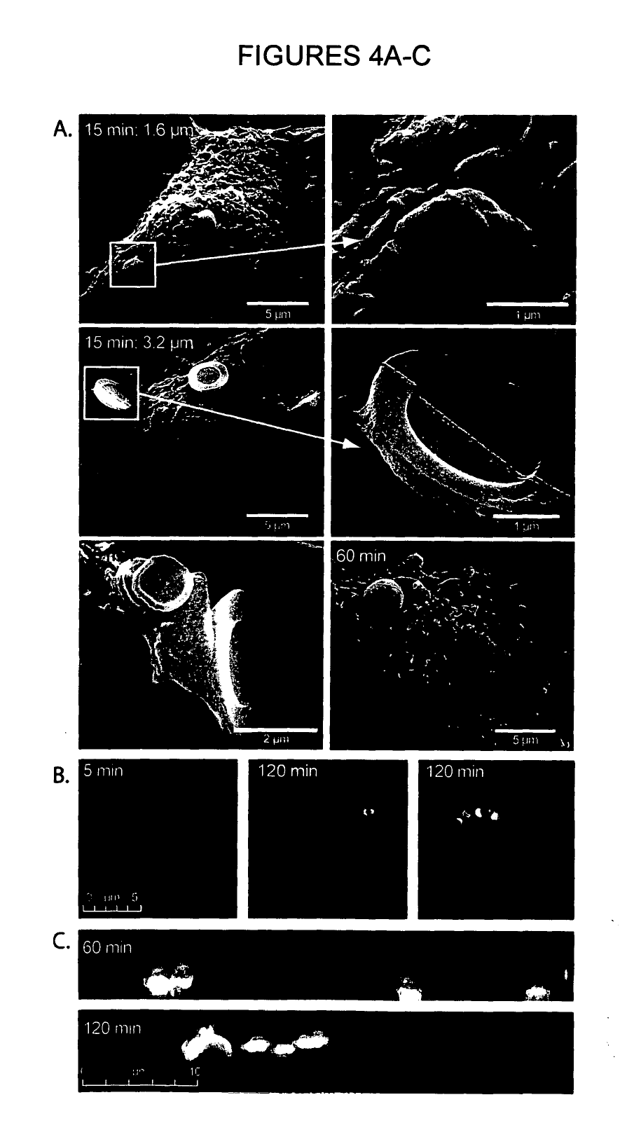

FIGS. 5A-C relate to an early uptake of oxidized silicon particles and FITC dextran by HUVECs (serum-free). FIGS. 5A and 5B are transmission electron micrographs showing HUVEC uptake of either 1.6 μm (A) or 3.2 μm (B) silicon particles after incubation at 37° C. for 15 min. FIG. 5C shows results of flow cytometric analysis of FITC Dextran internalization by HUVECs incubated for 1 hr with no particles (solid green peak, second from the left), 1.6 μm (red open peak, the right peak), or 3.2 μm (purple open peak, second from the right) silicon particles. The solid blue peak (the left peak) represents HUVECs incubated in media without FITC Dextran. In FIG. 5C, the x-axis is fluorescence due to internalized FITC dextran and the y-axis is counts (the height is dependent on the number of cells).

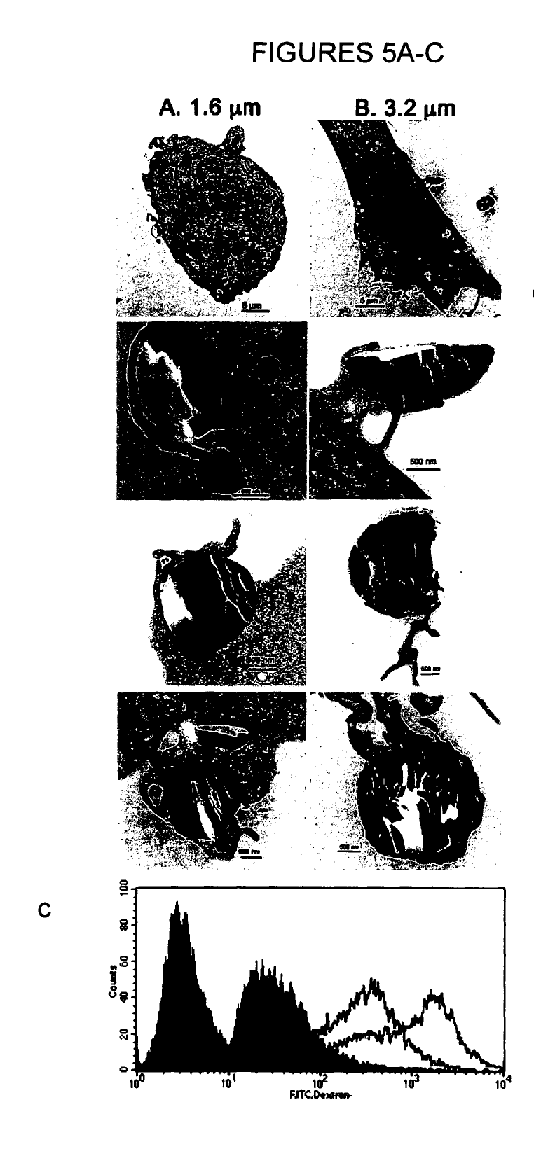

FIGS. 6A-B demonstrate cellular location of internalized particles at 2 hrs. FIG. 6A shows that smaller 1.6 μm particles are located in the perinuclear region of the cell. Membranes can be seen surrounding some of the particles. FIG. 6B shows that larger 3.2 μm particles are more scattered and lack apparent membranes, which may be indicative of endosomal escape. The resolution scale bar is 10 microns for major images in FIGS. 6A and 6B and 500 nm for insets.

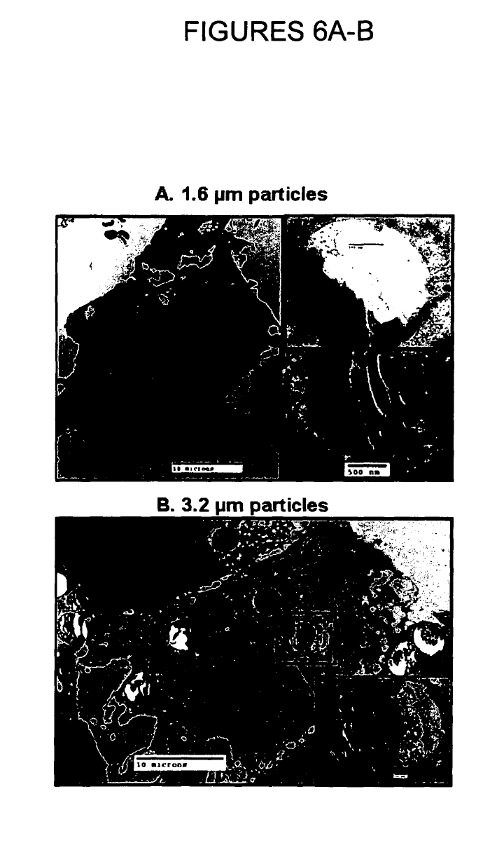

### DETAILED DESCRIPTION

### Related Applications

The following research articles and patent documents, which are all incorporated herein by reference in their entirety, may be useful for understanding the present inventions:

1) PCT publication no. WO 2007/120248 published Oct. 25, 2007;

2) PCT publication no. WO 2008/041970 published Apr. 10, 2008;

3) PCT publication no. WO 2008/021908 published Feb. 21, 2008;

4) U.S. Patent Application Publication no. 2008/0102030 published May 1, 2008;

5) U.S. Patent Application Publication no. 2003/0114366 published Jun. 19, 2003;

6) U.S. Patent Application Publication no. 2008/0206344 published Aug. 28, 2008;

7) U.S. Patent Application Publication no. 2008/0280140 published Nov. 13, 2008;

8) Tasciotti E. et al, 2008 Nature Nanotechnology 3, 151-157.

### Definitions

Unless otherwise specified “a” or “an” means one or more.

“Microparticle” means a particle having a maximum characteristic size from 1 micron to 1000 microns, or from 1 micron to 100 microns. “Nanoparticle” means a particle having a maximum characteristic size of less than 1 micron.

“Opsonin” is a protein that, when bound to a particle, increases the particle's phagocytosis.

“Dysopsonin” is a protein that, when bound to a particle, decreases the particle's phagocytosis.

“Opsonizable” refers to a particle, that can undergo opsonization when exposed to the blood or a blood component, such as serum, i.e. the particle that can bind one or more proteins from the blood or its component. Preferably, when exposed to the blood or a blood component, the opsonizable particle binds one or more opsonins and does not bind dysopsonins.

“Nanoporous” or “nanopores” refers to pores with an average size of less than 1 micron.

“Biodegradable” refers to a material that can dissolve or degrade in a physiological medium or a biocompatible polymeric material that can be degraded under physiological conditions by physiological enzymes and/or chemical conditions.

“Biocompatible” refers to a material that, when exposed to living cells, will support an appropriate cellular activity of the cells without causing an undesirable effect in the cells such as a change in a living cycle of the cells; a change in a proliferation rate of the cells and a cytotoxic effect.

### Disclosure

The present inventors discovered that opsonizable micro- or nanoparticles, that have a positive surface charge, can undergo opsonization in blood or a blood component, such as serum, in such a manner that the particles can preferentially bind proteins, that can allow the particles, after undergoing opsonization, to specifically target inflamed cells in a body of a subject. Preferably, prior to the opsonization, the positively charged opsonizable micro- or nanoparticles do not contain targeting ligands, such as antibodies, peptides and/or aptamers, disposed on their surface.

After undergoing opsonization, the positively charged opsonizable micro- or nanoparticles can also have a lower uptake by immune cells, such as macrophages, compared to otherwise identical micro or nanoparticles, which have, prior to opsonization, a negative surface charge or no surface charge. In the present context, the lower uptake can mean that it can take a longer time for the opsonized positively charged particles to be internalized by the immune cells than for the opsonized negatively charged particles or the neutral ones. As the result, the opsonized positively charged particles can avoid an uptake by immune cells in the body of the subject, when targeting the inflamed cells.

Preferably, prior to opsonization, a surface of the opsonizable particle does not contain an anti-opsonization coating, such as a coating formed by polyethylene glycol (PEG) or other hydrophilic chains. Although coating particles with hydrophilic chains, known as PEGylation, may reduce or prevent a rapid internatization of the particles by macrophages, at the same time PEGylation can often prevent particles from binding to target cell(s).

In many embodiments, the surface of the opsonizable particles, prior to the opsonization, does not contain albumin. In many embodiments, the surface of the opsonizable particles particles, prior to the opsonization, does not contain any opsonins. In many embodiments, the surface of the opsonizable particle, prior to the opsonization, does not contain any proteins.

The positively charged opsonizable particles can be used for treating, preventing and/or monitoring a condition associated with an inflammation, such as cytokine stimulated inflammation, in a subject, such as an animal with a blood system, by specifically targeting inflamed cells in the body of the subject. In many embodiments, the subject can be a mammal, such as a human.

The positively charged opsonizable particles can be used for specifically targeting inflamed vasculature and thereby for treating, preventing and/or monitoring a condition or disease associated with an inflammation.

Examples of such conditions include, but not limited to, allergies, asthma, Alzheimer's disease, diabetes, hormonal imbalances, autoimmune diseases, such as rheumatoid arthritis and psoriasis, osteoarthritis, osteoporosis, atherosclerosis, including coronary artery disease, vasculitis, chronic inflammatory conditions, such as obesity, ulcers, such as Marjolin's ulcer, respiratory inflammations caused by asbestos or cigarette smoke, foreskin inflammations, inflammations caused by viruses, such as Human papilloma virus, Hepatitic B or C or Ebstein-Barr virus, Schistosomiasis, pelvic inflammatory disease, ovarian epitheal inflammation, Barrett's metaplasia, H. pylori gastritis, chronic pancreatitis, Chinese liver fluke infestation, chronic cholecystitis and inflammatory bowel disease; and inflammation-associated cancers, which include prostate cancer, colon cancer, breast cancer; gastrointestinal tract cancers, such as gastric cancer, hepatocellular carcinoma, colorectal cancer, pancreatic cancer, gastric cancer, nasopharyngeal cancer, esophageal cancer, cholangiocarcinoma, gallbladder cancer and anogenital cancer; intergumentary cancer, such as skin carcinoma; respiratory tract cancers, such as bronchial cancer and mesothelioma; genitourinary tract cancer, such as phimosis, penile carcinoma and bladder cancer; reproductive system cancer, such as ovarian cancer.

In particular, the positively charged opsonizable particles can be used for preventing certain types by specifically targeting inflamed cells associated with an inflammatory condition, which can lead to the cancer. For example, by targeting inflammation caused by Majolin's ulcer, the positively charged opsonizable particles can prevent skin carcinoma; by targeting inflammation caused by asbestos, silica or smoking, the particles can prevent bronchial cancer; by targeting foreskin inflammation the particles can prevent phimosis; by targeting inflammation caused by Human papilloma virus the particles can prevent penile carcinoma and/or anogenital cancer; by targeting inflammation caused by Schistosomiasis the particles can prevent bladder cancer; by targeting inflammation caused by pelvic inflammatory disease or ovarian epithelial inflammation the particles can prevent ovarian cancer; by targeting inflammation caused by Ebstein-Barr virus the particles can prevent nasopharyngeal cancer; by targeting inflammation caused by Barrett's metaplasia the particles can prevent esophageal cancer; by targeting inflammation caused by H. pylori gastritis the particles can prevent gastric cancer; by targeting inflammation caused by chronic pancreatitis the particles can prevent pancreatic cancer; by targeting inflammation caused by Chinese liver fluke infestation the particles can prevent cholangiocarcinoma; by targeting inflammation caused by chronic cholecyctitis the particles can prevent gallbladder cancer; by targeting inflammation caused by Hepatitis B or C the particles can prevent hepacellucar carcinoma; by targeting inflammation caused by inflammatory bowel disease the particles can prevent colorectal cancer.

Conditions and diseases associated with an inflammation are disclosed in the following references: 1) M. Macarthur et al. Am. J. Physiol Gastrointest Livel Physiol. 286″ G515-520, 2004; 2) Calogero et al. Breast Cancer Research, v. 9(4), 2007; Wienberg et al. J. Clin. Invest, 112: 1796-1808, 2003; Xu et. al. J. Clin Invest, 112:1821-1830, 2003.

The positively charged opsonizable particles can be used as a part of a multistage drug delivery system disclosed in U.S. patent application no. US2008280140 and in PCT publication no. WO2008021908. For example, in some embodiments, the positive charged opsonizable particles can contain at least one second stage particle which can comprise an active agent.

### PARTICLE

The opsonizable particle can have a variety of shapes and sizes.

The dimensions of the opsonizable particle are not particularly limited and depend on an application. For example, for intravascular administration, a maximum characteristic size of the particle can be smaller than a radius of the smallest capillary in a subject, which is about 4 to 5 microns for humans.

In some embodiments, the maximum characteristic size of the particle may be less than about 100 microns or less than about 50 microns or less than about 20 microns or less than about 10 microns or less than about 5 microns or less than about 4 microns or less than about 3 microns or less than about 2 microns or less than about 1 micron. Yet in some embodiments, the maximum characteristic size of the particle may be from 100 nm to 3 microns or from 200 nm to 3 microns or from 500 nm to 3 microns or from 700 nm to 2 microns.

Yet in some embodiments, the maximum characteristic size of the particle may be greater than about 2 microns or greater than about 5 microns or greater than about 10 microns. The shape of the particle is not particularly limited. In some embodiments, the particle can be a spherical particle. Yet in some embodiments, the particle can be a non-spherical particle. In some embodiments, the particle can have a symmetrical shape. Yet in some embodiments, the particle can have an asymmetrical shape.

In some embodiments, the particle can have a selected non-spherical shape configured to facilitate a contact between the particle and a surface of the target site, such as endothelium surface of the inflamed vasculature. Examples of appropriate shapes include, but not limited to, an oblate spheroid, a disc or a cylinder. In some embodiments, the particle can be such that only a portion of its outer surface defines a shape configured to facilitate a contact between the particle and a surface of the target site, such as endothelium surface, while the rest of the outer surface does not. For example, the particle can be a truncated oblate spheroidal particle.

The dimensions and shape of particle that can facilitate a contact between the particle and a surface of the target site can be evaluated using methods disclosed in U.S. Patent Application Publication no. 2008/0206344 and U.S. application Ser. No. 12/181,759 filed Jul. 29, 2008.

In many embodiments, the opsonizable particle can be a porous particle, i.e. a particle that comprises a porous material. The porous material can be a porous oxide material or a porous etched material. Examples of porous oxide materials include, but no limited to, porous silicon oxide, porous aluminum oxide, porous titanium oxide and porous iron oxide. The term “porous etched materials” refers to a material, in which pores are introduced via a wet etching technique, such as electrochemical etching. Examples of porous etched materials include porous semiconductors materials, such as porous silicon, porous germanium, porous GaAs, porous InP, porous SiC, porous SiₓGe_{1-31 x}, porous GaP, porous GaN. Methods of making porous etched particles are disclosed, for example, U.S. Patent Application Publication No. 2008/0280140.

In many embodiments, the porous particle can be a nanoporous particle.

In some embodiments, a average pore size of the porous particle may be from about 1 nm to about 1 micron or from about 1 nm to about 800 nm or from about 1 nm to about 500 nm or from about 1 nm to about 300 nm or from about 1 nm to about 200 nm or from about 2 nm to about 100 nm.

In some embodiments, the average pore size of the porous particle can be no more than 1 micron or no more than 800 nm or more than 500 nm or more than 300 nm or no more than 200 nm or no more than 100 nm or no more than 80 nm or no more than 50 nm.

In some embodiments, the average pore size of the porous particle can be size from about 5 to about 100 nm or about 10 to about 60 nm or from about 20 to about 40 nm or from about 30 nm to about 30 nm.

In some embodiments, the average pore size of the porous particle can be from about 1 nm to about 10 nm or from about 3 nm to about 10 nm or from about 3 nm to about 7 nm.

In general, pores sizes may be determined using a number of techniques including N₂ adsorption/desorption and microscopy, such as scanning electron microscopy.

In some embodiments, pores of the porous particle may be linear pores. Yet in some embodiments, pores of the porous particle may be sponge like pores.

In some embodiments, at least one of the porous particle may comprise a biodegradable region. In many embodiments, the whole particle may be biodegradable.

In general, porous silicon may be bioinert, bioactive or biodegradable depending on its porosity and pore size. Also, a rate or speed of biodegradation of porous silicon may depend on its porosity and pore size, see e.g. Canham, Biomedical Applications of Silicon, in Canham LT, editor. Properties of porous silicon. EMIS datareview series No. 18. London: INSPEC. p. 371-376. The biodegradation rate may also depend on surface modification. Porous silicon particles and methods of their fabrication are disclosed, for example, in Cohen M. H. et al Biomedical Microdevices 5:3, 253-259, 2003; U.S. patent application publication No. 2003/0114366; U.S. Pat. Nos. 6,107,102 and 6,355,270; U.S. Patent Application Publication No. 2008/0280140; PCT publication no. WO 2008/021908; Foraker, A. B. et al. Pharma. Res. 20 (1), 110-116 (2003); Salonen; J. et al. Jour. Contr. Rel. 108, 362-374 (2005). Porous silicon oxide particles and methods of their fabrication are disclosed, for example, in Paik J. A. et al. J. Mater. Res., Vol 17, Aug 2002, p. 2121.

The opsonizable particles may be prepared using a number of techniques.

In some embodiments, the opsonizable particle may be a top-down fabricated particle, i.e. a particle produced utilizing a top-down microfabrication or nanofabrication technique, such as photolithography, electron beam lithography, X-ray lithography, deep UV lithography, nanoimprint lithography or dip pen nanolithography. Such fabrication methods may allow for a scaled up production of particles, that are uniform or substantially identical in dimensions.

### ACTIVE AGENT

The active agent can be a therapeutic agent, an imaging agent or a combination thereof. The active agent can be an agent that can be released from a particle containing it. The selection of the active agent depends on the application.

### THERAPEUTIC AGENT

The therapeutic agent may be any physiologically or pharmacologically active substance that can produce a desired biological effect in a targeted site in an animal, such as a mammal or a human. The therapeutic agent may be any inorganic or organic compound, without limitation, including peptides, proteins, nucleic acids, and small molecules, any of which may be characterized or uncharacterized. The therapeutic agent may be in various forms, such as an unchanged molecules, molecular complexe, pharmacologically acceptable salt, such as hydrochloride, hydrobromide, sulfate, laurate, palmitate, phosphate, nitrite, nitrate, borate, acetate, maleate, tartrate, oleate, salicylate, and the like. For acidic therapeutic agent, salts of metals, amines or organic cations, for example, quaternary ammonium, can be used.

Derivatives of drugs, such as bases, esters and amides also can be used as a therapeutic agent. A therapeutic agent that is water insoluble can be used in a form that is a water soluble derivative thereof, or as a base derivative thereof, which in either instance, or by its delivery, is converted by enzymes, hydrolyzed by the body pH, or by other metabolic processes to the original therapeutically active form.

The therapeutic agent can be a chemotherapeutic agent, an immunosuppressive agent, a cytokine, a cytotoxic agent, a nucleolytic compound, a radioactive isotope, a receptor, and a pro-drug activating enzyme, which may be naturally occurring or produced by synthetic or recombinant methods, or any combination thereof.

Drugs that are affected by classical multidrug resistance, such as vinca alkaloids (e.g., vinblastine and vincristine), the anthracyclines (e.g., doxorubicin and daunorubicin), RNA transcription inhibitors (e.g., actinomycin-D) and microtubule stabilizing drugs (e.g., paclitaxel) can have particular utility as the therapeutic agent.

A cancer chemotherapy agent may be a preferred therapeutic agent. Useful cancer chemotherapy drugs include nitrogen mustards, nitrosorueas, ethyleneimine, alkane sulfonates, tetrazine, platinum compounds, pyrimidine analogs, purine analogs, antimetabolites, folate analogs, anthracyclines, taxanes, vinca alkaloids, topoisomerase inhibitors and hormonal agents. Exemplary chemotherapy drugs are Actinomycin-D, Alkeran, Ara-C, Anastrozole, Asparaginase, BiCNU, Bicalutamide, Bleomycin, Busulfan, Capecitabine, Carboplatin, Carboplatinum, Carmustine, CCNU, Chlorambucil, Cisplatin, Cladribine, CPT-11, Cyclophosphamide, Cytarabine, Cytosine arabinoside, Cytoxan, Dacarbazine, Dactinomycin, Daunorubicin, Dexrazoxane, Docetaxel, Doxorubicin, DTIC, Epirubicin, Ethyleneimine, Etoposide, Floxuridine, Fludarabine, Fluorouracil, Flutamide, Fotemustine, Gemcitabine, Herceptin, Hexamethylamine, Hydroxyurea, Idarubicin, Ifosfamide, Irinotecan, Lomustine, Mechlorethamine, Melphalan, Mercaptopurine, Methotrexate, Mitomycin, Mitotane, Mitoxantrone, Oxaliplatin, Paclitaxel, Pamidronate, Pentostatin, Plicamycin, Procarbazine, Rituximab, Steroids, Streptozocin, STI-571,Streptozocin, Tamoxifen, Temozolomide, Teniposide, Tetrazine, Thioguanine, Thiotepa, Tomudex, Topotecan, Treosulphan, Trimetrexate, Vinblastine, Vincristine, Vindesine, Vinorelbine, VP-16, and Xeloda.

Useful cancer chemotherapy drugs also include alkylating agents, such as Thiotepa and cyclosphosphamide; alkyl sulfonates such as Busulfan, Improsulfan and Piposulfan; aziridines such as Benzodopa, Carboquone, Meturedopa, and Uredopa; ethylenimines and methylamelamines including altretamine, triethylenemelamine, trietylenephosphoramide, triethylenethiophosphaoramide and trimethylolomelamine; nitrogen mustards such as Chlorambucil, Chlornaphazine, Cholophosphamide, Estramustine, Ifosfamide, mechlorethamine, mechlorethamine oxide hydrochloride, Melphalan, Novembiehin, Phenesterine, Prednimustine, Trofosfamide, uracil mustard; nitroureas such as Cannustine, Chlorozotocin, Fotemustine, Lomustine, Nimustine, and Ranimustine; antibiotics such as Aclacinomysins, Actinomycin, Authramycin, Azaserine, Bleomycins, Cactinomycin, Calicheamicin, Carabicin, Carminomycin, Carzinophilin, Chromoinycins, Dactinomycin, Daunorubicin, Detorubicin, 6-diazo-5-oxo-L-norleucine, Doxorubicin, Epirubicin, Esorubicin, Idambicin, Marcellomycin, Mitomycins, mycophenolic acid, Nogalamycin, Olivomycins, Peplomycin, Potfiromycin, Puromycin, Quelamycin, Rodorubicin, Streptonigrin, Streptozocin, Tubercidin, Ubenimex, Zinostatin, and Zorubicin; anti-metabolites such as Methotrexate and 5-fluorouracil (5-FU); folic acid analogues such as Denopterin, Methotrexate, Pteropterin, and Trimetrexate; purine analogs such as Fludarabine, 6-mercaptopurine, Thiamiprine, and Thioguanine; pyrimidine analogs such as Ancitabine, Azacitidine, 6-azauridine, Carmofur, Cytarabine, Dideoxyuridine, Doxifluridine, Enocitabine, Floxuridine, and 5-FU; androgens such as Calusterone, Dromostanolone Propionate, Epitiostanol, Rnepitiostane, and Testolactone; anti-adrenals such as aminoglutethimide, Mitotane, and Trilostane; folic acid replenisher such as frolinic acid; aceglatone; aldophosphamide glycoside; aminolevulinic acid; Amsacrine; Bestrabucil; Bisantrene; Edatraxate; Defofamine; Demecolcine; Diaziquone; Elfornithine; elliptinium acetate; Etoglucid; gallium nitrate; hydroxyurea; Lentinan; Lonidamine; Mitoguazone; Mitoxantrone; Mopidamol; Nitracrine; Pentostatin; Phenamet; Pirarubicin; podophyllinic acid; 2-ethylhydrazide; Procarbazine; PSK®; Razoxane; Sizofrran; Spirogermanium; tenuazonic acid; triaziquone; 2, 2′,2″-trichlorotriethylamine; Urethan; Vindesine; Dacarbazine; Mannomustine; Mitobronitol; Mitolactol; Pipobroman; Gacytosine; Arabinoside (“Ara-C”); cyclophosphamide; thiotEPa; taxoids, e.g., Paclitaxel (TAXOL®, Bristol-Myers Squibb Oncology, Princeton, N.J.) and Doxetaxel (TAXOTERE®, Rhone-Poulenc Rorer, Antony, France); Chlorambucil; Gemcitabine; 6-thioguanine; Mercaptopurine; Methotrexate; platinum analogs such as Cisplatin and Carboplatin; Vinblastine; platinum; etoposide (VP-16); Ifosfamide; Mitomycin C; Mitoxantrone; Vincristine; Vinorelbine; Navelbine; Novantrone; Teniposide; Daunomycin; Aminopterin; Xeloda; Ibandronate; CPT-11; topoisomerase inhibitor RFS 2000; difluoromethylornithine (DMFO); retinoic acid; Esperamicins; Capecitabine; and pharmaceutically acceptable salts, acids or derivatives of any of the above. Also included are anti-hormonal agents that act to regulate or inhibit hormone action on tumors such as anti-estrogens including for example Tamoxifen, Raloxifene, aromatase inhibiting 4(5)-imidazoles, 4 Hydroxytamoxifen, Trioxifene, Keoxifene, Onapristone, And Toremifene (Fareston); and anti-androgens such as Flutamide, Nilutamide, Bicalutamide, Leuprolide, and Goserelin; and pharmaceutically acceptable salts, acids or derivatives of any of the above.

Cytokines can be also used as the therapeutic agent. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β ; mullerian-inhibiting substance; mouse gonadotropin-associated peptide; inhibin; activin; vascular endothelial growth factor; integrin; thrombopoietin (TPO); nerve growth factors such as NGF-β; platelet growth factor; transforming growth factors (TGFs) such as TGF-α and TGF-β; insulin-like growth factor-I and -II; erythropoietin (EPO); osteoinductive factors; interferons such as interferon-α, -β and -γ, colony stimulating factors (CSFs) such as macrophage-CSF (M-CSF); granulocyte-macrophage-CSF (GM-CSF); and granulocyte-CSF (GCSF); interleukins (ILs) such as IL-1, IL-1a, IL-2, IL-3, IL-4, IL-5, IL-6, IL-7, IL-8, IL-9, IL-11, IL-12, IL-15; a tumor necrosis factor such as TNF-α or TNF-β; and other polypeptide factors including LIF and kit ligand (KL). As used herein, the tern cytokine includes proteins from natural sources or from recombinant cell culture and biologically active equivalents of the native sequence cytokines.

In some embodiments, the therapeutic agent can be an antibody-based therapeutic agent, such as herceptin.

In some embodiments, the therapeutic agent can be a nanoparticle. For example, in some embodiments, the nanoparticle can be a nanoparticle that can be used for a thermal oblation or a thermal therapy. Examples of such nanoparticles include iron and gold nanoparticles.

### IMAGING AGENT

The imaging agent can be any substance that can provide imaging information about a targeted site in a body of an animal, such as a mammal or a human being. The imaging agent can comprise a magnetic material, such as iron oxide or a gadolinium containing compound, for magnetic resonance imaging (MRI). For optical imaging, the active agent can be, for example, semiconductor nanocrystal or quantum dot. For optical coherence tomography imaging, the imaging agent can be metal, e.g. gold or silver, nanocage particles. The imaging agent can be also an ultrasound contrast agent, such as a micro or nanobubble or iron oxide micro or nanoparticle.

### ADMINISTRATION

The opsonizable micro or nanoparticle(s) can be administered as a part of a composition, that includes a plurality of the particles, to a subject, such as human, via a suitable administration method in order to treat, prevent and/or monitor a physiological condition, such as a disease. The opsonizable micro or nanoparticle(s) are administered in such a manner so that, upon the administration, the particles can undergo opsonization in the blood of the subject.

The particular method employed for a specific application can be determined by the attending physician. Typically, the composition can be administered by one of the following routes: topical, parenteral, inhalation/pulmonary, oral, vaginal and anal.

The particles can be particularly useful for oncological applications, i.e. for treatment and/or monitoring cancer or a condition, such as tumor associated with cancer.

The majority of therapeutic applications can involve some type of parenteral administration, which includes intravenous (i.v.), intramuscular (i.m.) and subcutaneous (s.c.) injection. Administration of the particles can be systemic or local. The non-parenteral examples of administration recited above are examples of local administration. Intravascular administration can be either local or systemic. Local intravascular delivery can be used to bring a therapeutic substance to the vicinity of a known lesion by use of guided catheter system, such as a CAT-scan guided catheter. General injection, such as a bolus i.v. injection or continuous/trickle-feed i.v. infusion are typically systemic.

Preferably, the composition containing opsonizable particles is administered via i.v. infusion, via intraductal administration or via intratumoral route.

The opsonizable particles can be formulated as a suspension that contains a plurality of the particles. Preferably, the particles are uniform in their dimensions and their content. To form the suspension, the particles as described above can be suspended in any suitable aqueous carrier vehicle. A suitable pharmaceutical carrier is one that is non-toxic to the recipient at the dosages and concentrations employed and is compatible with other ingredients in the formulation. Preparation of suspension of microfabricated particles is disclosed, for example, in U.S. patent application publication No. 20030114366.

Embodiments described herein are further illustrated by, though in no way limited to, the following working examples.

### EXAMPLE

Nanoporous hemispherical silicon microparticles were designed, engineered, and fabricated in the Microelectronics Research Center at The University of Texas at Austin. Two sizes of microparticles were generated, with mean diameters of 1.6±0.2 and 3.2±0.2 μm, and pore sizes ranging from either 5-10 or 30-40 nm (porosity can be altered for different applications). Processing details are disclosed in Tasciotti E. et al, 2008 Nature Nanotechnology 3, 151-157.

Briefly, heavily doped p++ type (100) silicon wafers with resistivity of 0.005 ohm-cm (Silicon Quest, Inc, Santa Clara, Calif.) were used as the silicon source. A 100 nm layer of low-stress silicon nitride was deposited using a Low Pressure Chemical Vapor Deposition (LPCVD) system. Standard photolithography was used to pattern the microparticles over the wafer using a contact aligner (EVG 620 aligner) and AZ5209 photoresist. Nitride on particle patterns was selectively removed by CF4 based reactive ion etching (RIE). After the photoresist was stripped in piranha solution, the wafer was placed in a home-made Teflon cell for two-step electrochemical etching. Firstly, the wafers were etched in a mixture of hydrofluoric acid (HF) and Ethanol (1:1 v/v) by applying a current density of 6 mA/cm² for 105 s for 3.2 μm particles or 40 s for 1.6 μm particles, respectively. Then a high porosity release layer was formed by changing the current density to 320 mA/cm² for 6 s in a 2:5 v/v mixture of HF and Ethanol. Finally, the nitride layer was removed in HF after etching, and microparticles were released by ultrasound in isopropyl alcohol (IPA) for 1 min. The IPA solution containing porous silicon microparticles was collected and stored at 4° C. The morphology of the microparticles was examined by SEM.

### OXIDATION OF SILICON MICROPARTICLES

Silicon microparticles in isopropyl alcohol (IPA) were dried in a glass beaker kept on a hot plate (110° C.). The dried microparticles were then treated with piranha solution (1 volume H₂O₂ and 2 volumes of H₂SO₄). The suspension was heated to 110-120° C. for 2 hr with intermittent sonication to disperse the microparticles. The suspension was then washed in deionized (DI) water until the pH of the suspension was ˜5.5 - 6.

### SURFACE MODIFICATION OF SILICON MICROPARTICLES WITH APTES

The oxidized microparticles were washed in IPA 3-4 times. They were then suspended in IPA containing 0.5% (v/v) APTES (Sigma) for 2 hr at room temperature. The APTES modified microparticles were washed and stored in IPA. APTES modification was evaluated by measuring the zeta potential and by colorimetric analysis of amine density. The later was found to correlate with zeta potential measurements.

### PEG CONJUGATION

APTES modified microparticles were reacted with 10 mM mPEG-SCM-5000 (methoxy poly-ethylene glycol succinimidyl carboxymethyl; purchased from Laysan Bio Inc) in acetonitrile for 1.5 hr. The microparticles were then washed in distilled water 4-6 times to remove any unreacted mPEG. Zeta potential measurements were used to indicate adequate surface coating.

1.6 μm and 3.2 μm silicon microparticles were oxidized with a piranha solution [30:70 (v/v); H₂O₂:H₂SO₄] to create negatively charged, hydroxylated microparticles. Next, the oxidized microparticles were surface modified with 3-aminopropyltriethoxysilane (APTES), which yielded positively charged, amine modified microparticles. APTES modified microparticles were further conjugated with PEG for comparison.

Overall, the following three types of silicon microparticles have been compared: 1) negatively charged hydroxylated microparticles; 2) positively charged, amino modified microparticles; 3) PEGylated microparticles.

Using human umbilical vein endothelial cells (HUVECs), which are known as a model for vascular endothelium, see Klein et al., Pathobiology, 1994, 62, 199-208, scanning electron microscope (SEM) images were taken of cells after incubation with microparticles. HUVEC were purchased from Lonza Walkersville, Inc (Walkersville, Md.) and were cultured in EBM®-2 medium (Clonetics®, CC-3156). Cells were maintained at 37° C. in a humidified 5% CO₂ atmosphere. HUVEC samples were sputter coated with a 10 nm layer of gold using a Plasma Sciences CrC-150 Sputtering System (Torr International, Inc.). SEM images were acquired under high vacuum, at 20.00 kV, spot size 3.0-5.0, using a FEI Quanta 400 FEG ESEM equipped with an ETD (SE) detector.

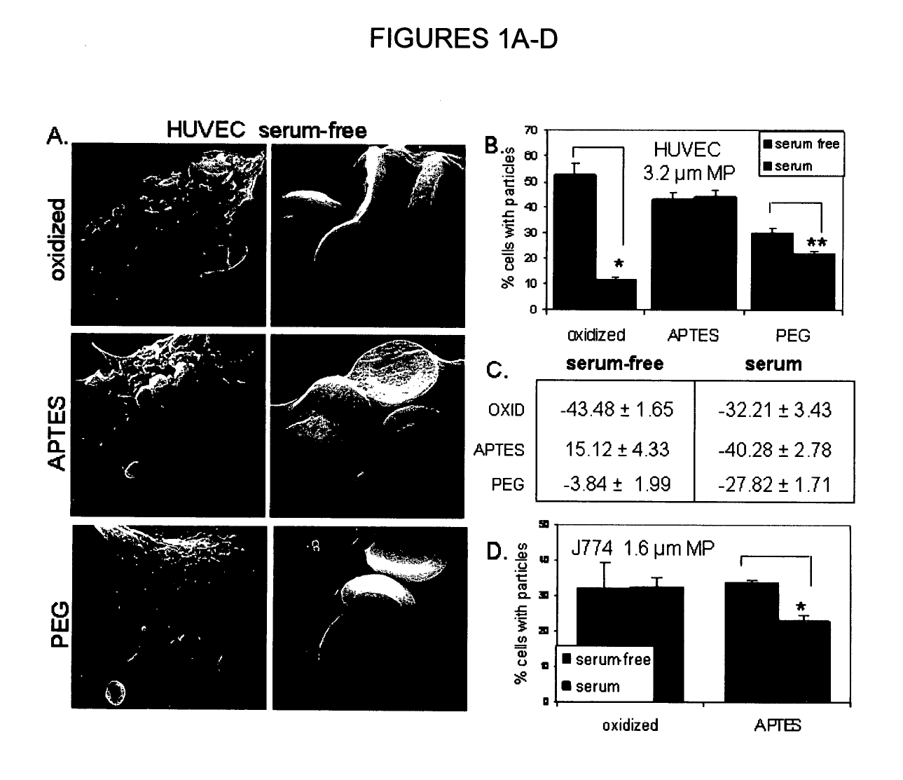

After one hour at 37° C., both positive and negative microparticles were internalized by HUVECs in serum-free media (FIG. 1A). While both positive and negative microparticles are internalized by HUVECs in serum-free media, it was surprisingly found that serum opsonization inhibits uptake of negative (oxidized) microparticles, without significantly affecting positively charged aminomodified particles.

For opsonization, particles were suspended in 100% serum for 1 hour on ice. Serum in the experiments was Fetal bovine serum from Clonetics®. Surface modification of silicon microparticles with PEG suppressed internalization of microparticles by HUVECs (FIG. 1B). In FIG. 1B, the y-axis is a percentage with internalized particles. Internalization experiments in FIG. 1B were performed for 1 hour at 37 ° C. Ratio of cell to particles was 1 cell per 20 particles in each of the experiments.

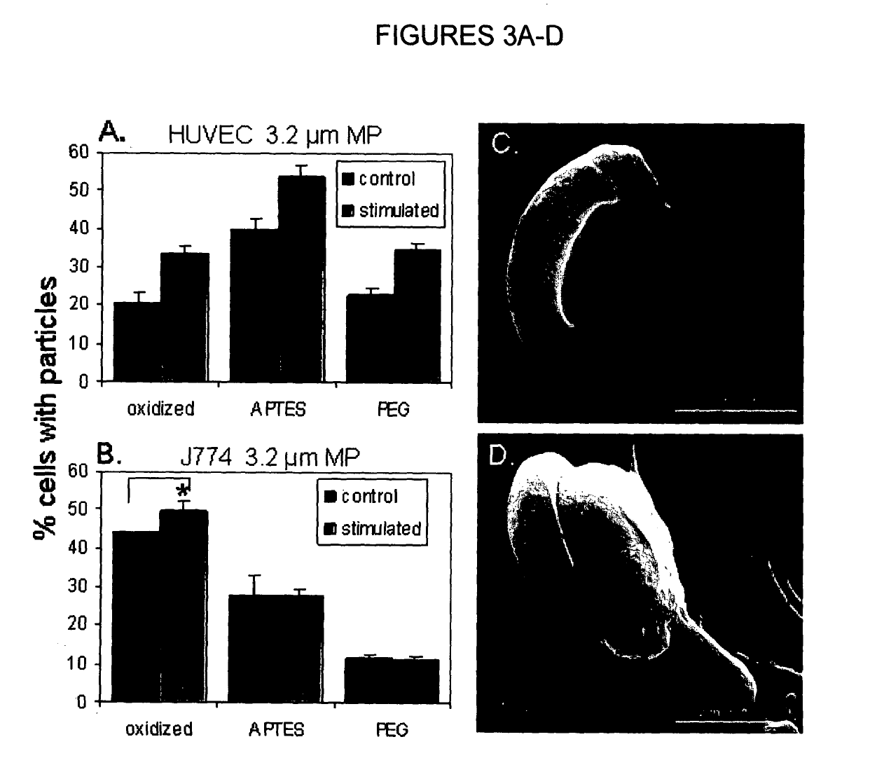

Activation of endothelial cells by pro-inflammatory cytokines can alter expression of cell surface receptors and thus can alter binding to particles, see Klein et al., Pathobiology, 1994, 62, 199-208. Endothelial cells (HUVECs) were stimulated with cytokines [TNF-α (10 ng/ml) and IFN-γ (100 U/ml), both obtained from Invitrogen] for 48 hrs. Subsequently, the stimulated HUVECs incubated with silicon particles, either negative (oxidized) or positive [amine (APTES)-modified] particles, following serum opsonization of the particles. Internalization of serum opsonized silicon microparticles by HUVECs was enhanced for all groups of microparticles following exposure to TNF-α and IFN-γ; however a clear preference for opsonized positive microparticles continued to exist, see FIG. 3A. In contrast to endothelial cells, macrophages (J774 cells) preferentially interacted with serum-opsonized negative microparticles. This preference for opsonized negative, oxidized microparticles by macrophages was significantly enhanced (11%) in the presence of cytokines (p=0.045), see FIG. 3B. On the other hand, uptake of APTES and PEG modified microparticles by macrophages was not affected by exposure to TNF-α and IFN-γ.

The experiments on HUVECS and J774 cells exposed to TNF-α and IFN-γ were performed as follows:

HUVECs (1.5×10⁵ cells/well) were seeded into 6 well plates and 24 hr later the cells were incubated with serum opsonized silicon microparticles (20 microparticles/cell) for 1 hr. at 37° C. Cells were then washed with PBS, harvested by trypsinization (HUVEC) or scrapping (J774), and resuspended in PBS containing 1.0% BSA and 0.1% sodium azide (FACS wash buffer). Microparticle association with cells was determined by measuring side scatter using a Becton Dickinson FACSCalibur Flow equipped with a 488-nm argon laser and CellQuest software (Becton Dickinson; San Jose, Calif.). Data is presented as the percentage of cells with microparticles (percent of cells with high side scatter). Side scatter due to cells in the absence of particles has been subtracted from the presented data.

J774A.1 macrophage cells were purchased from American Type Culture Collection (Manassas, Va.). Growth medium was Dulbecco's Modified Eagle's Medium containing 10% FBS, 100 μg/ml streptomycin and 100 U/ml Penicillin (Invitrogen; Carlsbad, Calif.). Cells were collected by scrapping.

Diagrams 3C-D are SEM images of silicon microparticle uptake by HUVEC (C) and J774 (D) cells (30 min, 37° C.) in the presence of serum. Cells were plated in 24 well plates containing 5×7 mm Silicon Chip Specimen Supports (Ted Pella, Inc., Redding, Calif.) at 5×10⁴ cells per well. When cells were confluent, media containing microparticles (1:10, cell:microparticles, 0.5 ml/well) was introduced and cells were incubated at 37° C. for the 30 min. Samples were washed with PBS and fixed in 2.5% glutaraldehyde for 30 min (Sigma-Aldrich; St. Louis, Mo.). After washing in PBS, cells were dehydrated in ascending concentrations of ethanol (30%, 50%, 70%, 90%, 95%, and 100%) for 10 min each.

HUVECs were then incubated in 50% alcohol-hexamethyldisilazane (Sigma) solution for 10 min followed by incubation in pure HMDS for 5 min to prepare for overnight incubation in a desiccator. Specimens were mounted on SEM stubs (Ted Pella, Inc.) using conductive adhesive tape (12 mm OD PELCO Tabs, Ted Pella, Inc.). Samples were sputter coated with a 10 nm layer of gold using a Plasma Sciences CrC-150 Sputtering System (Ton International, Inc.). SEM images were acquired under high vacuum, at 20.00 kV, spot size 3.0-5.0, using a FEI Quanta 400 FEG ESEM equipped with an ETD (SE) detector.

This research can suggest that vascular targeting of endothelial cells can be enhanced by serum opsonins that preferentially bind to positively charged microparticles. In contrast, serum opsonins binding to negatively charged microparticles strongly inhibit uptake by endothelial cells. Fortunately, professional phagocytes, such as macrophages, showed a preference for negatively charged opsonized microparticles. Although the present inventions are in no way limited by a theory, it can be suggested that opsonins binding to negatively charged microparticles can be reflective of serum components, which can decorate bacteria and apopotic cells, both of which have a net negative surface charge and can be targets for uptake by neutrophils and macrophages, see e.g. Fadok, V. A. et al. J. Immunol. 148, 2207-2216 (1992) and Dickson, J. S. & Koohmaraie, M. Appl. Environ. Microbiol. 55, 832-836 (1989). Directing microparticle uptake through directed serum opsonization can resist the need for PEGylation and concurrent compromised targeting and altered degradation rates.

Microparticle internalization by endothelial cells can be enhanced by pro-inflammatory cytokine stimulation, supporting superior uptake of positively charged microparticles at sites of chronic inflammation. Thus, opsonized microengineered particles with a positive surface charge can preferentially targeting of endothelium associated with inflamed pathologies, such as coronary artery disease, vasculitis, and cancer.

### ADDITIONAL REFERENCES

1. Campos S. The oncologist 2003;8 Suppl 2:10-6.

2. Lyass O, Uziely B, Ben-Yosef R, et al. Cancer 2000;89:1037-47.

3. Valero V. Oncology (Williston Park) 2002;16:35-43.

4. Blum JL, Savin MA, Edelman G, et al. Clinical breast cancer 2007;7:850-6.

5. Gradishar WJ. Expert Opin Pharmacother 2006;7: 1041-53.

6. Iyer AK, Khaled G, Fang J, Maeda H. Drug Discov Today 2006;11:812-8.

7. U.S. patent application publication no. 20070237827.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

What is claimed is:

1. A method for treating or monitoring a condition associated with an inflammation, comprising administering to a subject in need thereof a composition comprising opsonizable micro- or nanoparticles, that contain at least one active agent, wherein a surface of the micro or nanoparticles a) has a positive electrical charge and b) does not contain targeting ligands.

2. The method of claim 1, wherein the inflammation is a cytokine stimulated inflammation.

3. The method of claim 1, wherein the condition is a coronary artery disease.

4. The method of claim 1, wherein the condition is vasculitis.

5. The method of claim 1, wherein the condition is cancer.

6. The method of claim 1, wherein the administering is performed intravascularly.

7. The method of claim 1, wherein the subject is a human.

8. The method of claim 1, wherein the composition is a suspension comprising the opsonizable micro- or nanoparticles.

9. The method of claim 1, wherein the surface of the micro- or nanoparticles does not contain hydrophilic polymer chains.

10. The method of claim 1, wherein the micro- or nanoparticles are micro- or nanofabricated particles.

11. The method of claim 1, wherein the micro or nanoparticles are porous particles.

12. The method of claim 11, wherein the micro- or nanoparticles are nanoporous particles.

13. The method of claim 11, wherein the micro or nanoparticles are silicon porous particles.

14. The method of claim 11, wherein the micro- or nanoparticles are oxide porous particles.

15. The method of claim 14, wherein the micro- or nanoparticles are silicon oxide porous particles.

16. The method of claim 1, wherein the surface of the micro or nanoparticles is an aminomodified surface.

17. The method of claim 16, wherein the surface of the micro or nanoparticles is aminomodified by an aminosilane.

18. The method of claim 1, wherein said active agent is a therapeutic agent.

19. The method of claim 1, wherein said active agent is an imaging agent.

20. The method of claim 1, wherein said administering results in opsonization of said micro or nanoparticles and in targeting of cells associated with the inflammation by the opsonized micro- or nanoparticles.

21. The method of claim 20, wherein the cells associated with the inflammation are endothelial cells.

22. The method of claim 20, wherein the opsonized micro- or nanoparticles avoid uptake by macrophages of the subject.

23. A composition comprising opsonizable micro- or nanoparticles, that contain at least one active agent, wherein a surface of the micro or nanoparticles a) has a positive electrical charge and b) does not contain targeting ligands.

24. The composition of claim 23, further comprising a solution and wherein the micro- or nanoparticles are suspended in the solution.

25. The composition of claim 23, wherein the surface of the micro- or nanoparticles does not contain hydrophilic polymer chains.

26. The composition of claim 23, wherein the micro- or nanoparticles are micro- or nanofabricated particles.

27. The composition of claim 23, wherein the micro or nanoparticles are porous particles.

28. The composition of claim 27, wherein the micro- or nanoparticles are nanoporous particles.

29. The composition of claim 27, wherein the micro or nanoparticles are silicon porous particles.

30. The composition of claim 27, wherein the micro- or nanoparticles are oxide porous particles.

31. The composition of claim 30, wherein the micro- or nanoparticles are silicon oxide porous particles.

32. The composition of claim 23, wherein the surface of the micro- or nanoparticles is an aminomodified surface.

33. The composition of claim 32, wherein the surface of the micro- or nanoparticles is modified by an aminosilane.

34. The composition of claim 23, wherein said active agent is a therapeutic agent.

35. The composition of claim 23, wherein said active agent is an imaging agent.

36. A kit comprising the composition of claim 23.

37. A method for targeting inflamed cells in a subject,
comprising administering to the subject a composition comprising opsonizable micro- or nanoparticles, that contain at least one active agent, wherein a surface of the micro or nanoparticles a) has a positive electrical charge and b) does not contain targeting ligands.
